(19) United States
(12) Patent Application Publication
(10) Pub. No.: US 2026/0152453 A1
(43) Pub. Date: Jun. 4, 2026

(54) APPARATUS AND METHOD FOR GAS-LIQUID SEPARATION OF A LIQUID, GAS AND WATER VAPOUR MIXTURE FROM A FISCHER-TROPSCH REACTOR

(71) Applicant: Johnson Matthey Davy Technologies Limited, London (GB)

(72) Inventors: Robert Miles BAKER, London (GB); Paul Robert TICEHURST, London (GB)

(21) Appl. No.: 19/121,675

(22) PCT Filed: Nov. 22, 2023
(86) PCT No.: PCT/GB2023/053046

§ 371 Date: Apr. 16, 2025

(30) Foreign Application Priority Data
Dec. 8, 2022 (GB) 2218484.0

(51) Int. Cl. C07C 1/02; B01D 19/00; B01D 53/00

(52) U.S. Cl. CPC C07C 1/02; B01D 19/0042; B01D 53/002

(57) ABSTRACT

A method comprising the steps of feeding a product stream comprising a mixture of liquid and gaseous hydrocarbon products and water vapour from a Fischer-Tropsch reactor through a sequence of first, second, third and fourth vapour-liquid separators inside which the product stream is separated into different liquids and gas streams, wherein the feed to the third vapour-liquid separator is at a first temperature and the feed to the fourth vapour-liquid separator is at a second temperature, and the second temperature is less than the first temperature and less than or equal to 50° C.

### FIELD OF THE INVENTION

The invention relates to apparatus and methods for gas-liquid separation, and in particular for separating a product stream that comprises hydrocarbon products and water vapour into separate liquid and gas fractions.

### BACKGROUND OF THE INVENTION

It is known to produce oxygenated organic chemicals by means of chemical reaction. One well-known process for producing hydrocarbons at industrial scale is the Fischer-Tropsch process in which a mixture of carbon monoxide and hydrogen are reacted in the presence of a catalyst in a reactor to produce reaction products comprising hydrocarbons. The temperature and pressure used in the Fischer-Tropsch process are such that the reaction products initially discharged from the reactor are a mixture of liquid and gaseous hydrocarbon products and water vapour.

It is desirable to further process the reaction products to separate wax products from lighter hydrocarbon products. Typically, this further processing requires cooling the reactant products. A problem that can occur is that such cooling may cause the wax products to solidify and be deposited on components of the apparatus, for example in cooling apparatus such as condensers. This can lead to blockage of the apparatus necessitating the shut-down of the apparatus to allow for removal of the waxy deposits. Shutting down the reactor and restarting it leads to inefficiency, for example due to lost processing time and additional maintenance costs.

WO2019/016757 describes an apparatus and method for separating wax products from the products of an isothermal or adiabatic fixed bed reactor configured to carry out the Fischer-Tropsch process. WO2019/016757 attempts to address the problem of wax deposits by providing a first condenser and a second condenser that are in parallel fluid communication with the isothermal or adiabatic fixed bed reactor. In use, product from the Fischer-Tropsch process is first passed through the first condenser where it is cooled resulting in wax product solidifying and being collected in the first condenser. Once a predetermined amount of wax product has been collected in the first condenser, the product flow is switched to flow the product through the second condenser allowing the first condenser to be emptied of the wax product offline. Therefore, the use of the parallel first and second condensers permits continuous treatment of the product. However, the solution proposed in WO2019/016757 necessitates the use of parallel condensers, only one of which is ever in use at one time, which leads to inefficiency and additional complication, capital expenditure and maintenance costs.

WO2016185334 discloses the use of many-trayed distillation columns to produce various tight cuts of low carbon hydrocarbon products from a Fischer-Tropsch product stream. The method comprises: a) providing a first product stream comprising a C2-C3 hydrocarbon stream, a C4 hydrocarbon stream comprising butane and butene, and a C5+ hydrocarbon stream; b) separating at least a portion of the C2-C3 hydrocarbon stream from the first product stream; c) separating at least a portion of the C4 hydrocarbon stream comprising butane and butene from the first product stream; d) separating at least a portion of the C5+ hydrocarbon stream from the first product stream; and e) converting at least a portion of the butene in the C4 hydrocarbon stream to butadiene.

The present invention seeks to tackle at least some of the problems associated with the prior art or at least to provide a commercially acceptable alternative solution thereto.

### SUMMARY OF THE INVENTION

One aspect of the present disclosure is directed to a method comprising the steps of:
a) feeding a product stream comprising a mixture of liquid and gaseous hydrocarbon products and water vapour from a Fischer-Tropsch reactor into a first vapour-liquid separator inside which the product stream is separated into a first liquid comprising a first cut of the hydrocarbon products and a first gas stream comprising gaseous hydrocarbon products and water vapour;
b) collecting the first liquid at a liquid outlet of the first vapour-liquid separator;
c) discharging the first gas stream from a gas outlet of the first vapour-liquid separator and feeding the first gas stream through a first cooler to apply cooling to the first gas stream to condense a portion of the gaseous hydrocarbon products and water vapour to form a first cooled mixture;
d) feeding the first cooled mixture from the first cooler into a second vapour-liquid separator inside which the first cooled mixture is separated into a second liquid comprising a second cut of the hydrocarbon products and water, and a second gas stream comprising gaseous hydrocarbon products and water vapour;
e) collecting the second liquid at a liquid outlet of the second vapour-liquid separator;
f) discharging the second gas stream from a gas outlet of the second vapour-liquid separator;
g) feeding the second gas stream from the gas outlet of the second vapour-liquid separator through a second cooler to apply cooling to the second gas stream to condense a portion of the gaseous hydrocarbon products and water vapour to form a second cooled mixture;
h) feeding at a first temperature the second cooled mixture from the second cooler into a third vapour-liquid separator inside which the second cooled mixture is separated into a third liquid comprising a third cut of the hydrocarbon products and water, and a third gas stream comprising gaseous hydrocarbon products and water vapour;
i) collecting the third liquid at a liquid outlet of the third vapour-liquid separator;
j) discharging the third gas stream from a gas outlet of the third vapour-liquid separator;
k) feeding the third gas stream from the gas outlet of the third vapour-liquid separator through a third cooler to apply cooling to the third gas stream to condense a portion of the remainder of the hydrocarbon products and water vapour to form a third cooled mixture;
l) feeding at a second temperature the third cooled mixture from the third cooler into a fourth vapour-liquid separator inside which the third cooled mixture is separated into a fourth liquid comprising a fourth cut of the hydrocarbon products and water, and a fourth gas stream;
m) collecting the fourth liquid at a liquid outlet of the fourth vapour-liquid separator; and
n) discharging the fourth gas stream from a gas outlet of the fourth vapour-liquid separator,
wherein the second temperature is less than the first temperature and less than or equal to 50° C.

Another aspect of the present disclosure is directed to an apparatus coupled to the outlet of a Fischer-Tropsch reactor comprising:
a first vapour-liquid separator comprising:
   an inlet for receiving a product stream comprising hydrocarbon products and water vapour;
   a liquid outlet for discharging a first liquid comprising a first cut of the hydrocarbon products; and
   a gas outlet for discharging a first gas stream comprising a remainder of the hydrocarbon products and water vapour;
a first cooler positioned in between the first vapour-liquid separator and a second vapour-liquid separator for cooling the first gas stream to form a first cooled mixture;
the second vapour-liquid separator comprising:
   an inlet for receiving the first cooled mixture;
   a liquid outlet for discharging a second liquid comprising a second cut of the hydrocarbon products and water; and
   a gas outlet for discharging a second gas stream comprising a remainder of the hydrocarbon products and water vapour;
a second cooler positioned in between the second vapour-liquid separator and a third vapour-liquid separator for cooling the second gas stream to form a second cooled mixture;
the third vapour-liquid separator comprising:
   an inlet for receiving the second cooled mixture;
   a liquid outlet for discharging a third liquid comprising a third cut of the hydrocarbon products and water; and
   a gas outlet for discharging a third gas stream comprising a remainder of the hydrocarbon products and water vapour;
a third cooler positioned in between the third vapour-liquid separator and a fourth vapour-liquid separator for cooling the third gas stream to form a third cooled mixture; and
the fourth vapour-liquid separator comprising:
   an inlet for receiving the third cooled mixture;
   a liquid outlet for discharging a fourth liquid comprising a fourth cut of the hydrocarbon products and water; and
   a gas outlet for discharging a fourth gas stream.

The present method and system provide a sequence of processing units in order to produce four (rough) cuts of hydrocarbon products from a Fischer-Tropsch reactor. The sequence of processing units is: a first vapour-liquid separator; a first cooler; a second vapour-liquid separator; a second cooler; a third vapour-liquid separator; a third cooler; and a fourth vapour-liquid separator. Such a sequential configuration beneficially reduces or substantially eliminates the build-up of deposits of wax products in a vapour-liquid separator and/or cooler unit. The present scheme does not require the use of dedicated distillation schemes to split the product into tight cuts, but rather provides a staged cooling train to produce at least four rough cuts. That is, the present scheme provides a sequential liquid knock-out scheme to segregate Fischer-Tropsch products into different mixed liquids with different handling and/or further processing requirements without the use of complex distillation column technology.

### BRIEF DESCRIPTION OF THE DRAWINGS

FIG. 1 is a diagram of an apparatus according to the present invention suitable for carrying out the method of the present invention.

FIG. 2 is a graph showing the final boiling point and mass % of total FT liquids in the crude naphtha product for various air cooler exit temperatures with a water-cooling exit temperature of 30° C.

FIG. 3 is a graph showing the final boiling point and mass % of total FT liquids in the crude naphtha product for various air cooler exit temperatures with a water-cooling exit temperature of 40° C.

FIG. 4 is a graph showing the final boiling point and mass % of total FT liquids in the crude naphtha product for various air cooler exit temperatures with a water-cooling exit temperature of 47° C.

### DETAILED DESCRIPTION OF THE INVENTION

In a first aspect, the present disclosure is directed to a method comprising the steps of:
a) feeding a product stream comprising a mixture of liquid and gaseous hydrocarbon products and water vapour from a Fischer-Tropsch reactor into a first vapour-liquid separator inside which the product stream is separated into a first liquid comprising a first cut of the hydrocarbon products and a first gas stream comprising gaseous hydrocarbon products and water vapour;
b) collecting the first liquid at a liquid outlet of the first vapour-liquid separator;
c) discharging the first gas stream from a gas outlet of the first vapour-liquid separator and feeding the first gas stream through a first cooler to apply cooling to the first gas stream to condense a portion of the gaseous hydrocarbon products and water vapour to form a first cooled mixture;
d) feeding the first cooled mixture from the first cooler into a second vapour-liquid separator inside which the first cooled mixture is separated into a second liquid comprising a second cut of the hydrocarbon products and water, and a second gas stream comprising gaseous hydrocarbon products and water vapour;
e) collecting the second liquid at a liquid outlet of the second vapour-liquid separator;
f) discharging the second gas stream from a gas outlet of the second vapour-liquid separator;
g) feeding the second gas stream from the gas outlet of the second vapour-liquid separator through a second cooler to apply cooling to the second gas stream to condense a portion of the gaseous hydrocarbon products and water vapour to form a second cooled mixture;
h) feeding at a first temperature the second cooled mixture from the second cooler into a third vapour-liquid separator inside which the second cooled mixture is separated into a third liquid comprising a third cut of the hydrocarbon products and water, and a third gas stream comprising gaseous hydrocarbon products and water vapour;
i) collecting the third liquid at a liquid outlet of the third vapour-liquid separator;
j) discharging the third gas stream from a gas outlet of the third vapour-liquid separator;
k) feeding the third gas stream from the gas outlet of the third vapour-liquid separator through a third cooler to apply cooling to the third gas stream to condense a portion of the remainder of the hydrocarbon products and water vapour to form a third cooled mixture;
l) feeding at a second temperature the third cooled mixture from the third cooler into a fourth vapour-liquid separator inside which the third cooled mixture is separated into a fourth liquid comprising a fourth cut of the hydrocarbon products and water, and a fourth gas stream;
m) collecting the fourth liquid at a liquid outlet of the fourth vapour-liquid separator; and
n) discharging the fourth gas stream from a gas outlet of the fourth vapour-liquid separator,
wherein the second temperature is less than the first temperature and less than or equal to 50° C.

Each aspect or embodiment as defined herein may be combined with any other aspect(s) or embodiment(s) unless clearly indicated to the contrary. In particular, any features indicated as being preferred or advantageous may be combined with any other feature indicated as being preferred or advantageous.

Advantageously, the method may allow for continuous operation. The method may beneficially reduce or substantially eliminate the build-up of deposits of wax products in the vapour-liquid separators and/or the coolers, for example by configuring the first vapour-liquid separator to function as a wax trap. The method may utilise at least four vapour-liquid separators and at least three coolers that are arranged in series. Such an arrangement may thereby avoid the need to duplicate equipment in a parallel configuration. In addition, such an arrangement may allow for three or more cuts (e.g. liquid fractions) to be separated from the gas stream in a controlled and flexible manner. In particular, such an arrangement may permit multistage separation and cooling to be applied to the product stream. In particular, the separation of the product stream may be carried out in stages, with a first stage functioning to remove wax products from the product stream while maintaining the temperature of the product stream sufficiently hot to prevent or at least substantially reduce the deposit of wax products on the walls or components of the apparatus. The staged cooling may be configured to separate desirable cuts of hydrocarbon products at temperatures where each cut is maintained as a mobile, liquid phase.

The term “vapour-liquid separator” as used herein may encompass a device configured to separate liquids from a gas. Examples of such vapour-liquid separators may be referred to as knockout pots, knockout drums or flash drums. The separation of the liquid from the gas within the vapour-liquid separator may be achieved by one or more mechanisms, used alone or in combination, including for example gravitational separation, velocity reduction, directional changes (including centrifugal separation), and impingement on elements such as vanes, demister pads and filter elements. The vapour-liquid separator may comprise a drum (or other vessel) that may be vertically or horizontally arranged. It is to be noted that the vapour-liquid separators are not distillation columns and especially not complex multi-trayed distillation columns used to produce tight cuts of hydrocarbon product.

The term “cooler” as used herein may encompass a device configured to applying cooling to a medium, in particular a gas and/or a liquid. Examples of such coolers include condensers and interchangers. The coolers may utilise, for example, one or more of gas cooling and liquid cooling. The liquid for cooling may comprise cooling water. The coolers may be configured as a produced water cooler so as to reduce or eliminate flash evaporation during pressure reduction of the liquid. The coolers may be configured as co-current coolers. The gas for cooling may comprise air. Alternatively, the gas may comprise a feed gas mixture for the Fischer-Tropsch reactor that produces, in use, the product stream.

Beneficially, the first and second coolers may use different heat exchange mediums. For example, the first cooler may use a feed gas mixture for the Fischer-Tropsch reactor as its heat exchange medium and the second cooler may use cooling gas as its heat exchange medium. Beneficially, a gradual and staged cooling of the product stream may be enabled by using a relatively hot heat exchange medium in the first cooler and a relatively cold heat exchange medium in the second cooler. The second cooler may use a colder heat exchange medium (e.g. air) because the remaining, lighter, hydrocarbons by that point will remain mobile and liquid at the lower wall temperatures in that portion of the apparatus, since the less mobile hydrocarbons (e.g. wax products and first cut products) have already been removed.

Beneficially, the second and third coolers may use different heat exchange mediums. For example, the second cooler may use cooling gas as its heat exchange medium, such as air, and the third cooler may use cooling liquid as its heat exchange medium, such as water. Beneficially, a gradual and staged cooling of the product stream may be enabled by using a relatively hot heat exchange medium in the second cooler and a relatively cold heat exchange medium in the third cooler. The third cooler may use a colder heat exchange medium (e.g. water) because the remaining, lighter, hydrocarbons by that point will remain mobile and liquid at the lower wall temperatures in that portion of the apparatus, since the less mobile hydrocarbons (e.g. wax products, first cut products and second cut products) have already been removed.

Advantageously, the method may result in the fourth liquid comprising a naphtha cut. By “naphtha cut”, it is meant a cut comprising C3 to C9 hydrocarbons (that is, hydrocarbons molecules containing three to nine carbon atoms), more specifically a cut comprising primarily C3 to C9 hydrocarbons. Such a naphtha cut may be recycled to a syngas generation unit for generating syngas for the Fischer-Tropsch unit. This may increase the carbon efficiency of the method.

The second and third cut products will typically be passed to an upgrading unit. Since the naphtha cut is removed from such products, it does not need to be carried through the upgrading unit, meaning that the upgrading is more energy efficient and/or the upgrading unit may be smaller and/or less complex.

Surprisingly, the separation of the naphtha cut may occur with only very little loss in the other cuts of the hydrocarbon products, typically substantially no loss in the other cuts of the hydrocarbon products (i.e. the second cut, third cut).

The second temperature is preferably from 20 to 50° C., more preferably from 25 to 45° C. Lower temperatures may be hard to achieve using conventional coolers. Higher temperatures may result in less naphtha being recovered in the fourth liquid and more being present in the fourth gas stream. In addition, higher temperatures may result in the fourth liquid exhibiting an unfavourably high final boiling point (FBP), for example a FBP above 240° C. FBPs higher than about 240° C. may render the fourth liquid more difficult to process within a syngas generation unit.

The first temperature is preferably less than or equal to 75° C., more preferably less than or equal to 70° C. This may help to ensure a low FBP of the fourth liquid, for example a FBP not higher than 240° C. Without being bound by theory, it is considered that this is because a higher first temperature will result in more higher boiling point species from remaining gaseous, and thereby forming part of the third gas stream rather than the third liquid. In addition, higher temperatures may result in higher levels of product loss. In other words, higher temperatures may result in a higher proportion of hydrocarbons (non-naphtha hydrocarbons) that might otherwise be present in the third liquid/third cut instead remaining gaseous and forming part of the third gas stream.

The first temperature is preferably more than or equal to 50° C., more preferably more than or equal to 55° C., even more preferably more than or equal to 60° C. Such temperatures may increase the amount of naphtha contained in the fourth liquid (that may be recycled to a syngas generation unit) and decrease the amount of naphtha contained in the third liquid (which would need to be carried through an upgrading unit).

Preferably, the first temperature is greater than the second temperature by at least 10° C., more preferably at least 15° C. This may reduce the FBP of the fourth liquid. Without being bound by theory, it is considered that smaller differences between the first and second temperatures may result in only heavier hydrocarbons condensing, and thereby the FBP of the fourth liquid actually increasing.

The third vapour-liquid separator and the fourth vapour-liquid separator preferably operate at a pressure of less than 55 bar(a), more preferably less than 50 bar(a). This may reduce the FBP of the fourth liquid. Without being bound by theory, it is considered that higher pressures result in a higher level of condensation of hydrocarbon species at a given temperature.

Accordingly, more lighter hydrocarbons condense to form the fourth liquid, thereby decreasing its FBP.

Preferably, either: the third vapour-liquid separator and the fourth vapour-liquid separator operate at a pressure of less than 50 bara, or the second temperature is less than or equal to 70° C. This may reduce the FBP of the fourth liquid.

The first cut of the hydrocarbon products may comprise or consist of a wax product. The wax product may have a carbon chain length of C20 and higher. The term “wax product” as used herein may encompass a hydrocarbon having at least 20 carbon atoms. For example, the wax product may be a C20-C120 hydrocarbon, such as for example a C20-C40 hydrocarbon, a C20-C60 hydrocarbon, a C20-C80 hydrocarbon, or a C20-C110 hydrocarbon.

Beneficially, the first cut of the hydrocarbon products may be separated from the gas stream before the gas stream is passed through the first cooler. Arranging for the first cut to comprise or consist of a wax product may allow for the wax product to be separated from the gas stream at an early stage of the process while the gas stream is still relatively hot, thereby reducing or substantially eliminating the likelihood of deposits of waxy hydrocarbon product blocking the apparatus, especially any cooler of the apparatus.

The method may comprise using a supplemental cooler to cool the second liquid and/or third liquid before it reaches the decanter.

The second cut of the hydrocarbon products may be a heavier cut than the third cut of the hydrocarbon products. The second cut of the hydrocarbon products may comprise C5 to C30 hydrocarbons and the third cut of the hydrocarbon products may comprise C3 to C18 hydrocarbons.

The method may comprise feeding the fourth liquid from the liquid outlet of the fourth vapour-liquid separator into a decanter inside which the fourth liquid is separated into liquid hydrocarbon products and water.

The term “decanter” as used herein may encompass a device configured for separating at least two liquid phases from each other, for example a hydrocarbon liquid phase from a liquid water phase. The decanter may be arranged horizontally, vertically or be spherical. The decanter may have means for venting a gas phase.

The fourth cut preferably comprises C3 to C9 hydrocarbons. Such species may be recycled to a syngas generation unit to generate syngas for the Fischer Tropsch reactor.

The decanter may be operated at the same pressure as the Fischer-Tropsch reactor or lower, for example, at a pressure of less than 10 bar(a); optionally less than 8 bar(a); optionally at 3 to 7 bar(a).

In some embodiments, the method may further comprise cooling the product stream before it reaches the first vapour-liquid separator by passing the product stream through a pre-cooler. Advantageously, use of the pre-cooler may enable control of a feed temperature of the product stream to the first vapour-liquid separator. This may be particularly beneficial where the incoming product stream may vary in temperature. The Fischer-Tropsch process uses a catalyst that ages over time. The catalyst ageing may necessitate increasing the reactor temperature over time to compensate. Thus, the temperature of the product stream may vary, e.g. increase, over time within a set period. The use of the pre-cooler may allow for accommodation of a variable temperature product stream. In particular the use of the pre-cooler may help to avoid carry-over of wax products to the first cooler by providing sufficient pre-cooling to the product stream such that the first vapour-liquid separator can still adequately function to remove the wax products from the product stream.

Preferably, the first and/or second cooler may be cooled by a feed gas mixture for the Fischer-Tropsch reactor.

More preferably, the product stream may be cooled before it reaches the first vapour-liquid separator in a pre-cooler in heat exchange with a feed gas mixture for the Fischer-Tropsch reactor, preferably a feed gas mixture that has passed through the first cooler in heat exchange with the first gas mixture.

The temperature of the product stream exiting the reactor may be, for example, 205 to 240° C.

The pre-cooler (where present) may cool the product stream to a temperature in the range of 120 to 200° C., preferably 160 to 200° C.

The product stream may be fed to the first vapour-liquid separator at a temperature in the range of 120 to 200° C., preferably 160 to 200° C.

The first cooled mixture produced by the first cooler may have a temperature of, for example, 80 to 120° C.

The feed gas mixture may be heated to a temperature in the range of 100 to 180° C. in the first cooler.

The feed gas mixture may be heated to a temperature in the range of 190 to 220° C. in the pre-cooler (where present).

The first cooled mixture may be fed to the second vapour-liquid separator at a temperature in the range of 80 to 120° C.

The second cooled mixture produced by the second cooler may have a temperature of, for example, 55 to 75° C.

The second cooled mixture may be fed to the third vapour-liquid separator at a temperature in the range of, for example, 55 to 75° C.

The third cooled mixture produced by the third cooler may have a temperature of, for example, from 20 to 50° C.

The third cooled mixture may be fed to the third vapour-liquid separator at a temperature in the range of, for example, 20 to 50° C.

Advantageously, the use of the pre-cooler and/or the first cooler and/or the second cooler and/or the third cooler may enable controllable and flexible temperature control of the method. In particular, the temperatures of each of the vapour-liquid separators may be controllable independently of each other by co-ordinated use of the coolers, allowing for control of the temperature and thus the makeup of each liquid cut separated from the product stream. Beneficially this may enable prevention of carry-over of wax products to the first cooler as well as enabling the separation of desirable cuts of liquid hydrocarbon products without the need for distillation.

The method may further comprise passing the fourth liquid to a syngas generation unit to generate syngas for the Fischer-Tropsch reactor.

Another aspect of the present disclosure is directed to an apparatus coupled to the outlet of a Fischer-Tropsch reactor comprising:
a first vapour-liquid separator comprising:
   an inlet for receiving a product stream comprising hydrocarbon products and water vapour;
   a liquid outlet for discharging a first liquid comprising a first cut of the hydrocarbon products; and
   a gas outlet for discharging a first gas stream comprising a remainder of the hydrocarbon products and water vapour;
a first cooler positioned in between the first vapour-liquid separator and a second vapour-liquid separator for cooling the first gas stream to form a first cooled mixture;
the second vapour-liquid separator comprising:
   an inlet for receiving the first cooled mixture;
   a liquid outlet for discharging a second liquid comprising a second cut of the hydrocarbon products and water; and
   a gas outlet for discharging a second gas stream comprising a remainder of the hydrocarbon products and water vapour;
a second cooler positioned in between the second vapour-liquid separator and a third vapour-liquid separator for cooling the second gas stream to form a second cooled mixture;
the third vapour-liquid separator comprising:
   an inlet for receiving the second cooled mixture;
   a liquid outlet for discharging a third liquid comprising a third cut of the hydrocarbon products and water; and
   a gas outlet for discharging a third gas stream comprising a remainder of the hydrocarbon products and water vapour;
a third cooler positioned in between the third vapour-liquid separator and a fourth vapour-liquid separator for cooling the third gas stream to form a third cooled mixture; and
the fourth vapour-liquid separator comprising:
   an inlet for receiving the third cooled mixture;
   a liquid outlet for discharging a fourth liquid comprising a fourth cut of the hydrocarbon products and water; and
   a gas outlet for discharging a second gas stream.

The advantages and preferably features of the first aspect of the invention apply also to this aspect of the invention. The apparatus of this aspect may be used to perform the method of the first aspect.

Advantageously, the apparatus may be continually operated. The apparatus may be less prone to build-up of deposits of wax products in the vapour-liquid separators and/or the coolers. Beneficially the apparatus comprises at least four vapour-liquid separators and at least three coolers that are arranged in series. Such an arrangement may thereby avoid the need to duplicate equipment in a parallel configuration. In addition, such an arrangement may allow for one or more cuts (e.g. liquid fractions) to be separated from the product stream in a controlled and flexible manner. In particular, such an arrangement may permit multistage separation and cooling to be applied to the product stream. In particular, the separation of the product stream may be carried out in stages, with a first stage functioning to remove wax products from the product stream while maintaining the temperature of the product stream sufficiently hot to prevent or at least substantially reduce the deposit of wax products on the walls or components of the apparatus. The staged cooling may be configured to separate desirable cuts of hydrocarbon products at temperatures where each cut is maintained as a mobile, liquid phase.

The first cut of the hydrocarbon products may comprise or consist of a wax product.

Preferably the apparatus further comprises a decanter for separating liquid hydrocarbon products from water, the decanter comprising an inlet fluidly connected for receiving the fourth liquid from the fourth vapour-liquid separator, an outlet for liquid hydrocarbon products, and an outlet for water.

In some embodiments the apparatus further comprises a pre-cooler positioned upstream of the inlet of the first vapour-liquid separator for cooling the product stream before it reaches the first vapour-liquid separator.

Preferably the pre-cooler and the first cooler may be configured to be fed with a feed gas mixture for the Fischer-Tropsch reactor such that the product stream is cooled in heat exchange with the feed gas mixture after the feed gas mixture has passed through the first cooler in heat exchange with the first gas stream.

Preferably the, or each, vapour-liquid separator may comprise a tapered body comprising an upper portion having a relatively larger internal diameter for receiving a gas stream and a lower portion having a relatively smaller internal diameter for collecting liquid, wherein the liquid outlet of the vapour-liquid separator is located in the lower portion having the relatively smaller internal diameter. Beneficially, the tapered body may reduce and/or minimise the liquid residence time in the vapour-liquid separator which may in turn reduce or eliminate the hydrocarbon and water phases separating during flow towards the decanter.

### Examples

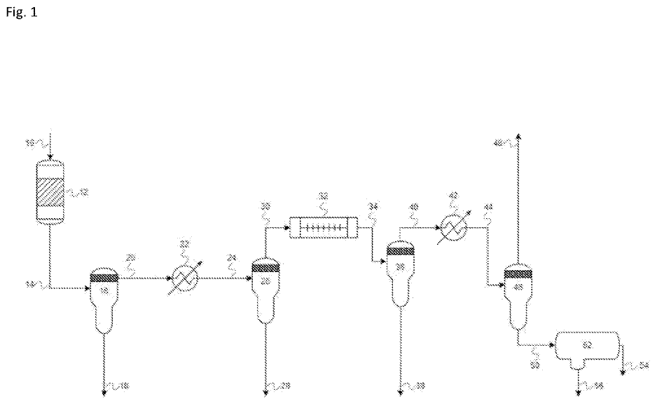

FIG. 1 shows a diagram of a first embodiment of apparatus according to the present invention suitable for carrying out the method of the present invention. The apparatus is configured for treating the reaction products output from a Fischer-Tropsch reactor. The Fischer-Tropsch reactor 12 is configured to receive a feedstock 10 and produce a product stream of reaction products 14 by reaction of the feedstock in the presence of a catalyst. The Fischer-Tropsch reactor 12 may carry out the Fischer-Tropsch process in which a feedstock of carbon monoxide and hydrogen are reacted in the presence of a catalyst to produce reaction products comprising hydrocarbons. The product stream of reaction products 14 initially discharged from the Fischer-Tropsch reactor 12 is a mixture of liquid and gaseous hydrocarbon products and water vapour. The temperature of the product stream exiting the Fischer-Tropsch reactor 12 may be, for example, 205 to 240° C.

The apparatus comprises a first vapour-liquid separator 16, a first cooler 22, a second vapour-liquid separator 26, a second cooler 32, a third vapour-liquid separator 36, a third cooler 42 and a fourth vapour-liquid separator 46 arranged in series. Further, a decanter 52 is provided in series and downstream of the fourth vapour-liquid separator 46.

The first vapour-liquid separator 16 comprises an inlet for receiving the product stream 14 that comprises the hydrocarbon products and water vapour output from the Fischer-Tropsch reactor 12, a liquid outlet for discharging a first liquid 18 comprising a first cut of the hydrocarbon products, and a gas outlet for discharging a first gas stream 20 comprising gaseous hydrocarbon products and water vapour. The first vapour-liquid separator 16 may be provided with a vane pack.

The first cooler 22 is positioned in between the first vapour-liquid separator 16 and the second vapour-liquid separator 26 and functions to cool the first gas stream 20 to produce a first cooled mixture 24 by condensing a portion of the gaseous hydrocarbon products and water vapour before it reaches the second vapour-liquid separator 26. The first cooler 22 may be configured to be fed with a feed gas mixture for the Fischer-Tropsch reactor 12 such that the first gas stream is cooled in heat exchange with the feed gas mixture.

The second vapour-liquid separator 26 comprises an inlet for receiving the first cooled mixture 24, a liquid outlet for discharging a second liquid 28 comprising a second cut of the hydrocarbon products and optionally water, and a gas outlet for discharging a second gas stream 30 comprising gaseous hydrocarbon products and water vapour.

The second cooler 32 is positioned in series in between the second vapour-liquid separator 26 and the third vapour-liquid separator 36 and functions to cool the second gas stream 30 to produce a second cooled mixture 34 by condensing a portion of the gaseous hydrocarbon products and water vapour before it reaches the third vapour-liquid separator 36.

The third vapour-liquid separator 36 comprises an inlet for receiving the second cooled mixture 34, a liquid outlet for discharging a third liquid 38 comprising a third cut of the hydrocarbon products and optionally water, and a gas outlet for discharging a third gas stream 40 comprising gaseous hydrocarbon products and water vapour.

The third cooler 42 is positioned in series in between the third vapour-liquid separator 36 and the fourth vapour-liquid separator 46 and functions to cool the third gas stream 40 to produce a third cooled mixture 44 by condensing a portion of the gaseous hydrocarbon products and water vapour before it reaches the fourth vapour-liquid separator 46.

The fourth vapour-liquid separator 46 comprises an inlet for receiving the third cooled mixture 44, a liquid outlet for discharging a fourth liquid 50 comprising a fourth cut of the hydrocarbon products and optionally water, and a gas outlet for discharging a fourth gas stream 48 comprising any remainder of the hydrocarbon products and water vapour, and which may be called a tail gas.

The decanter 52 comprises an inlet fluidly connected for receiving the fourth liquid 50 from the fourth vapour-liquid separator 46, an outlet for liquid hydrocarbon products 54, and an outlet for water 56. A gas outlet may be provided for venting of off-gases (not shown).

In use, the temperature of the product stream exiting the Fischer-Tropsch reactor 12 may be, for example, 205 to 240° C. The method comprises first feeding the product stream 14 into the first vapour-liquid separator 16. The product stream 14 may be fed to the first vapour-liquid separator 16 at a temperature in the range of 120 to 200° C., preferably 160 to 200° C. Inside the first vapour-liquid separator 16 the product stream is separated into the first liquid 18 comprising the first cut of the hydrocarbon products and the first gas stream 20 comprising gaseous hydrocarbon products and water vapour. The method also comprises collecting the first liquid at the liquid outlet of the first vapour-liquid separator 16. The first vapour-liquid separator 16 may function as a wax trap to capture and remove from the product stream wax products.

The method also comprises discharging the first gas stream 20 from the gas outlet of the first vapour-liquid separator 16 and feeding the first gas stream through the first cooler 22 to apply cooling to the first gas stream to cool the first gas stream to produce the first cooled mixture 24 by condensing a portion of the gaseous hydrocarbon products and water vapour. The first cooled mixture 24 produced by the first cooler 22 may have a temperature of, for example, 80 to 120° C. The first cooler 22 may use the feed gas mixture for the Fischer-Tropsch reactor 12 to cool the first gas stream by heat exchange between the feed gas mixture and the first gas stream. The feed gas mixture may be heated to a temperature in the range of 100 to 180° C. in the first cooler 22.

The method also comprises feeding the first cooled mixture 24 from the first cooler 22 into the second vapour-liquid separator 26. The first cooled mixture 24 may be fed to the second vapour-liquid separator 26 at a temperature in the range of 80 to 120° C. Inside the second vapour-liquid separator 26 the first cooled mixture 24 is separated into the second liquid 28 comprising the second cut of the hydrocarbon products and optionally water, and the second gas stream 30 comprising gaseous hydrocarbon products and water vapour.

The method also comprises collecting the second liquid 28 at the liquid outlet of the second vapour-liquid separator 26. The method also comprises discharging the second gas stream 30 from the gas outlet of the second vapour-liquid separator 26 and feeding the second gas stream 30 through the second cooler 32 to apply cooling to the second gas stream to produce the second cooled mixture 34 by condensing a portion of the gaseous hydrocarbon products and water vapour. The second cooled mixture 34 produced by the second cooler 32 may have a temperature of, for example, 55 to 75° C. The second cooler 32 may use cooling gas (e.g. air) in heat exchange with the second gas stream.

The method also comprises feeding the second cooled mixture 34 from the second cooler 32 into the third vapour-liquid separator 36. The second cooled mixture 34 may be fed to the third vapour-liquid separator 36 at a temperature in the range of 55 to 75° C. Inside the third vapour-liquid separator 36 the second cooled mixture 34 is separated into the third liquid 38 comprising the third cut of the hydrocarbon products and optionally water, and the third gas stream 40 comprising gaseous hydrocarbon products and water vapour.

The method also comprises collecting the third liquid 38 at the liquid outlet of the third vapour-liquid separator 36. The method also comprises discharging the third gas stream 40 from the gas outlet of the third vapour-liquid separator 36 and feeding the third gas stream through the third cooler 42 to apply cooling to the third gas stream to produce the third cooled mixture 44 by condensing a portion of the gaseous hydrocarbon products and water vapour. The third cooled mixture 44 produced by the third cooler 42 may have a temperature of, for example, 20 to 50° C. The third cooler 42 may use cooling liquid (e.g. water) in heat exchange with the third gas stream.

The method also comprises feeding the third cooled mixture 44 from the third cooler 42 into the fourth vapour-liquid separator 46. The third cooled mixture 44 may be fed to the fourth vapour-liquid separator 46 at a temperature in the range of 20 to 50° C. Inside the fourth vapour-liquid separator 46 the third cooled mixture 44 is separated into the fourth liquid 50 comprising the fourth cut of the hydrocarbon products and optionally water, and the fourth gas stream 48 comprising any remainder of the hydrocarbon products and water vapour. Further, the method also comprises collecting the fourth liquid 50 at the liquid outlet of the fourth vapour-liquid separator 46.

The fourth gas stream 48 may be relatively dry and may, for example, comprise tails gas (e.g. unreacted reactants, CO₂, methane, ethane, etc.) that may be recycled to the Fischer-Tropsch reactor 12, or otherwise discharged from the apparatus.

The method also comprises feeding the fourth liquid 50 into the decanter, inside which the fourth liquid is separated into a naphtha product 54 that is collected at outlet and water 56 that is collected at a separate outlet.

The method may further comprise sending the naphtha product 54 to a syngas generation unit (not shown) for generating syngas for the Fischer-Tropsch reactor 12.

It will be understood that the apparatus may comprise more than four vapour-liquid separators where desired. For example, additional pairs of a cooler and vapour-liquid separator may be added in series for further treatment of the fourth gas stream.

The apparatus shown in FIG. 1 was used to treat the reaction products output from a Fischer-Tropsch reactor. An air cooler was employed as the second cooler and a water cooler was employed as the third cooler. Varying the exit temperatures of the second and third coolers resulted in changes in the composition of the fourth liquid. Optimised conditions are shown in Table 1 below, where C1 represents organic molecules containing a single carbon atom, C2 represents organic molecules two carbon atoms, C3 represents organic molecules containing three carbon atoms, etc.

TABLE 1
Compositions of the fourth liquid for various exit temperatures of
the air cooler and water cooler (naphtha material corresponds to C9
or lower, whereas product material corresponds to C10 and above).

Second cooler 32 outlet
temperature (° C.)

| | | |
|---|---|---|
| 65 | 70 | 60 |

Third cooler 42 outlet
temperature (° C.)

| Mass % | 30 | 40 | 47 |
|---|---|---|---|
| C1 | 2.8 | 2.3 | 1.6 |
| C2 | 0.9 | 0.7 | 0.4 |
| C3 | 1.8 | 1.6 | 0.9 |
| C4 | 4.6 | 4.0 | 2.6 |
| C5 | 8.1 | 7.3 | 5.6 |
| C6 | 11.5 | 10.8 | 9.5 |
| C7 | 13.9 | 13.5 | 13.3 |
| C8 | 15.5 | 15.5 | 16.9 |
| C9 | 14.8 | 15.2 | 17.2 |
| C10 | 12.1 | 12.9 | 14.8 |
| C11 | 8.0 | 8.9 | 9.8 |
| C12 | 3.8 | 4.5 | 4.7 |
| C13 | 1.4 | 1.7 | 1.7 |
| C14 | 0.4 | 0.5 | 0.5 |
| C15+ | 0.0 | 0.1 | 0.1 |
| Final Boiling Point (° C.) | 236 | 240 | 240 |
| Mass % of naphtha range | 77% | 72% | 26% |
| material recovered to rough-cut | | | |
| naphtha stream | | | |
| Mass % of product range | 4% | 5% | 2% |
| material lost to rough-cut | | | |
| naphtha stream | | | |

Surprisingly, the method and apparatus of the present invention enabled recovery of a naphtha product with only minimal product loss.

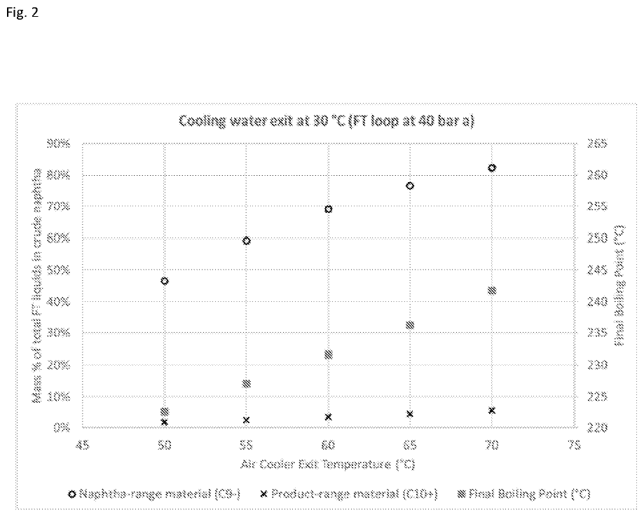

This impact of the cooling water temperature (third cooler 42 outlet) and effect of adjusting the air cooler temperatures (second cooler 32 outlet) are shown in the graphs of FIGS. 2 to 4.

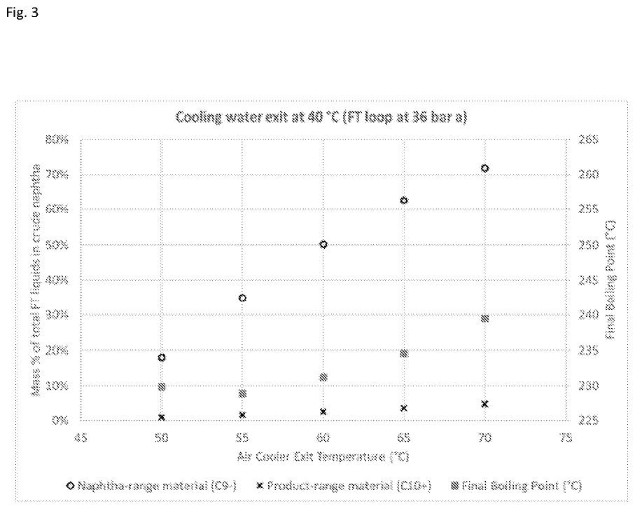

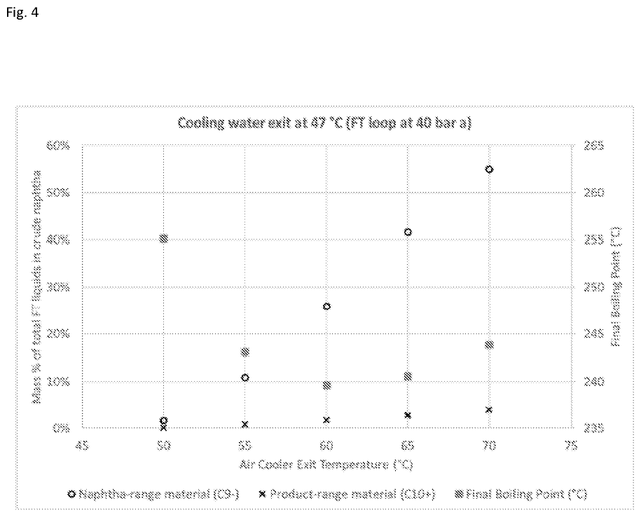

The general trend is that, as the cooling water temperature reduces, more of the naphtha range material can be recovered with minimal impact on the product range material. In general, as the air cooler temperature is increased, more naphtha range material slips to the cooling water exchanger and can be separated. However, this corresponds to an increase in heavier material being knocked out in the naphtha stream and so there is a limit on the FBP of the cut. The FT loop pressure for FIG. 3 system is 36 bar(a), whereas the FT loop pressure for the systems of FIGS. 2 and 4 is 40 bar(a). Accordingly, the FBP for a 70° C. air cooler exit is less than 240° C. for the 40° C.-cooling-water case but not the 30° C.-cooling-water case.

The foregoing detailed description has been provided by way of explanation and illustration, and is not intended to limit the scope of the appended claims. Many variations in the presently preferred embodiments illustrated herein will be apparent to one of ordinary skill in the art and remain within the scope of the appended claims and their equivalents.

1. A method comprising the steps of:
a) producing a product stream comprising a mixture of liquid and gaseous hydrocarbon products and water vapour using a Fischer-Tropsch reactor;
b) feeding product stream comprising the mixture of liquid and gaseous hydrocarbon products and water vapour from the Fischer-Tropsch reactor into a first vapour-liquid separator inside which the product stream is separated into a first liquid comprising a first cut of the hydrocarbon products and a first gas stream comprising gaseous hydrocarbon products and water vapour, wherein the product stream is fed to the first vapour-liquid separator at a temperature in a range of 120 to 200° C.;
c) collecting the first liquid at a liquid outlet of the first vapour-liquid separator;
d) discharging the first gas stream from a gas outlet of the first vapour-liquid separator and feeding the first gas stream through a first cooler to apply cooling to the first gas stream to condense a portion of the gaseous hydrocarbon products and water vapour to form a first cooled mixture;
e) feeding the first cooled mixture from the first cooler into a second vapour-liquid separator inside which the first cooled mixture is separated into a second liquid comprising a second cut of the hydrocarbon products and water, and a second gas stream comprising gaseous hydrocarbon products and water vapour, wherein the first cooled mixture is fed to the second vapour-liquid separator at a temperature in a range of 80 to 120° C.;
f) collecting the second liquid at a liquid outlet of the second vapour-liquid separator;
g) discharging the second gas stream from a gas outlet of the second vapour-liquid separator;
h) feeding the second gas stream from the gas outlet of the second vapour-liquid separator through a second cooler to apply cooling to the second gas stream to condense a portion of the gaseous hydrocarbon products and water vapour to form a second cooled mixture;
i) feeding at a first temperature the second cooled mixture from the second cooler into a third vapour-liquid separator inside which the second cooled mixture is separated into a third liquid comprising a third cut of the hydrocarbon products and water, and a third gas stream comprising gaseous hydrocarbon products and water vapour, wherein the first temperature is more than 50° C. and less than or equal to 75° C.;
j) collecting the third liquid at a liquid outlet of the third vapour-liquid separator;
k) discharging the third gas stream from a gas outlet of the third vapour-liquid separator;
l) feeding the third gas stream from the gas outlet of the third vapour-liquid separator through a third cooler to apply cooling to the third gas stream to condense a portion of the remainder of the hydrocarbon products and water vapour to form a third cooled mixture;
m) feeding at a second temperature the third cooled mixture from the third cooler into a fourth vapour-liquid separator inside which the third cooled mixture is separated into a fourth liquid comprising a fourth cut of the hydrocarbon products and water, and a fourth gas stream, wherein the second temperature is less than the first temperature, the second temperature being from 20 to 50° C.;
n) collecting the fourth liquid at a liquid outlet of the fourth vapour-liquid separator; and
o) discharging the fourth gas stream from a gas outlet of the fourth vapour-liquid separators.

2. The method of claim 1, wherein the second temperature is from 25 to 45° C.

3. The method of claim 1, wherein the first temperature is less than or equal to 70° C.

4. The method of claim 1, wherein the first temperature is more than or equal to 55° C., more preferably more than or equal to 60° C.

5. The method of claim 1, wherein the first temperature is greater than the second temperature by at least 10° C., preferably at least 15° C.

6. The method of claim 1, wherein the third vapour-liquid separator and the fourth vapour-liquid separator operate at a pressure of less than 55 bar(a), preferably less than 50 bar(a).

7. The method of claim 1, wherein the third vapour-liquid separator and the fourth vapour-liquid separator operate at a pressure of less than 50 bara.

8. The method of claim 1, wherein the first cut of the hydrocarbon products comprises or consists of a wax product having a carbon chain length of C20 and higher.

9. The method of claim 1, wherein the second cut of the hydrocarbon products is a heavier cut than the third cut of the hydrocarbon products; and the second cut of the hydrocarbon products comprises C5 to C30 hydrocarbons and the third cut of the hydrocarbon products comprises C3 to C18 hydrocarbons.

10. The method of claim 1, further comprising feeding the fourth liquid from the liquid outlet of the fourth vapour-liquid separator into a decanter inside which the fourth liquid is separated into liquid hydrocarbon products and water.

11. The method of claim 1, wherein the fourth cut comprises C3 to C9 hydrocarbons.

12. The method of claim 1, further comprising cooling the product stream before it reaches the first vapour-liquid separator by passing the product stream through a pre-cooler.

13. The method of claim 1, wherein the first and/or second cooler is cooled by a feed gas mixture for the Fischer-Tropsch reactor.

14. The method of claim 1, wherein the product stream is cooled before it reaches the first vapour-liquid separator in a pre-cooler in heat exchange with a feed gas mixture for the Fischer-Tropsch reactor, preferably a feed gas mixture that has passed through the first cooler in heat exchange with the first gas mixture.

15. (canceled)

16. The method of claim 1, further comprising passing the fourth liquid to a syngas generation unit to generate syngas for the Fischer-Tropsch reactor.

17-21. (canceled)
